(19) United States
(12) Patent Application Publication
(10) Pub. No.: US 2020/0138161 A1
(43) Pub. Date: May 7, 2020

(54) APPARATUS AND METHOD FOR CURLING EYELASHES

(71) Applicant: Kiesque, Incorporated, Wheaton, IL (US)

(72) Inventors: Jacqueline Agnes Smith, Wheaton, IL (US); Reid A. Baker, Aliso Viejo, CA (US)

(21) Appl. No.: 16/676,267

(22) Filed: Nov. 6, 2019

Related U.S. Application Data
(60) Provisional application No. 62/756,290, filed on Nov. 6, 2018.

(51) Int. Cl. A45D 2/48; A61K 8/81; A61Q 5/06; A45D 40/26

(52) U.S. Cl. CPC A45D 2/48; A61K 8/8111; A45D 2200/1036; A61Q 5/06; A45D 40/26; A61K 8/8164

(57) ABSTRACT

An eyelash curling device includes a mechanical eyelash curler and a carrier strip. The mechanical eyelash curler includes a first portion and a second portion configured to move relative to one another. The first portion includes an upper jaw. The second portion includes a lower jaw. The movement of the first and second portions relative to one another causes the upper and lower jaws to move from a first position in which the upper and lower jaws are located at a distance from one another and a second position in which the upper and lower jaws are in contact with one another. The carrier strip is coupled to at least one of the upper and lower jaws. The carrier strip includes at least one active agent therein on thereon. The at least one active agent is configured to aid in retaining a curl in eyelashes when it contacts the eyelashes.

### CROSS-REFERENCE TO RELATED APPLICATIONS

This application claims priority under 35 U.S.C. § 119 to U.S. Provisional Application No. 62/756,290, titled “Means for achieving a longer lasting curl for eyelashes,” and filed on Nov. 6, 2018. The contents of that application are hereby incorporated by reference in their entirety.

### TECHNICAL FIELD

This disclosure relates generally to cosmetic products, and more particularly, to devices and methods for curling eyelashes.

### BACKGROUND

Keeping eyelashes that have been curled using, e.g., a mechanical eyelash curling device, curled for a prolonged period of time is challenging to achieve. Often, one curls his/her eyelashes in traditional ways but a short time later finds that his/her eyelashes did not remain curled. Conventional devices for curling eyelashes do not give more than a temporary curl and/or are potentially dangerous.

Thus, a need exists for a safe a safe, efficient, and convenient way of maintaining an eyelash curl. The present disclosure is directed to solving these problems and addressing other needs.

### SUMMARY

According to some implementations of the present disclosure, an eyelash curling device includes a mechanical eyelash curler and a carrier strip. The mechanical eyelash curler includes a first portion and a second portion configured to move relative to one another. The first portion includes an upper jaw. The second portion includes a lower jaw. The movement of the first and second portions relative to one another causes the upper and lower jaws to move from a first position to a second position. The upper and lower jaws are located at a distance from one another in the first position. The upper and lower jaws are in contact with one another in the second position. The carrier strip is coupled to at least one of the upper and lower jaws. The carrier strip includes at least one active agent. The at least one active agent is configured to aid in retaining a curl or bend in eyelashes of a user when the at least one active agent contacts the eyelashes.

In some implementations, the carrier strip includes a flexible material having the at least one active agent. In some implementations, the carrier strip is configured to exude the least one active agent in response to pressure applied thereto by the upper and lower jaws in the second position. In some implementations, the at least one active agent is a non-soluble fixative agent.

In some implementations, the carrier strip is formed of an absorbent material configured to release a portion of the at least one active agent in response to pressure applied thereto by the upper and lower jaws in the second position.

In some implementations, the carrier strip is a flexible material having a fluid-activated composition including the at least one active agent thereon. In some such implementations, the fluid is water.

In some implementations, the eyelash curling device further includes a substantially airtight compartment for enclosing the carrier strip. In some such implementations, the carrier strip includes a solution comprising the at least one active agent thereon.

In some implementations, the eyelash curling device further includes a base strip coupled to at least one of the upper and lower jaws. The carrier strip is coupled to the base strip. In some such implementations, the carrier strip is removably coupled to the base strip. In some other such implementations, the carrier strip is adhesively coupled to the base strip.

In some implementations, the carrier strip includes a flexible material at least partially coated with the at least one active agent. In some implementations, the carrier strip includes a styrene-isoprene-styrene copolymer or a synthetic polyisoprene.

According to some implementations of the present disclosure, a carrier strip for use in enhancing or maintaining an eyelash curl includes a flexible base material and at least one active agent positioned on or within the base material. The at least one active agent is configured to aid in retaining the eyelash curl when the at least one active agent contacts the eyelashes. The carrier strip is configured to couple to at least one of an upper jaw and a lower jaw of a mechanical eyelash curler. The mechanical eyelash curler includes a first portion and a second portion configured to move relative to one another. The first portion includes the upper jaw. The second portion includes the lower jaw. The movement of the first and second portions relative to one another causes the upper and lower jaws to move from a first position to a second position. The upper and lower jaws are located at a distance from one another in the first position. The upper and lower jaws are in contact, via the carrier strip, with one another in the second position.

In some implementations, the flexible base material includes a matrix having the at least one active agent therein. The carrier strip is configured to exude the least one active agent in response to pressure applied thereto by the upper and lower jaws in the second position.

In some implementations, the carrier strip further includes a fluid-activated composition including the at least one active agent therein. The fluid-activated composition is applied on a surface of the flexible base material.

According to some implementations of the present disclosure, a method for retaining a curl or bend in eyelashes of a user is disclosed. The eyelashes of the user are positioned between an upper jaw and a lower jaw of a mechanical eyelash curler. The mechanical eyelash curler includes an upper jaw and a lower jaw. At least one of the upper and lower jaws includes a carrier strip coupled thereto. The carrier strip includes at least one active agent therein or thereon. The at least one active agent is configured to aid in retaining a curl or bend in eyelashes of a user when the at least one active agent contacts the eyelashes.

The upper jaw and the lower jaw of the mechanical eyelash curler are moved from a first position to a second position. In the first position, the upper and lower jaws are located at a distance from one another. In the second position, the upper and lower jaws are in contact with one another, and the eyelashes are sandwiched therebetween while contacting the carrier strip. The eyelashes of the user are curled or bent by maintaining the mechanical eyelash curler in the second position such that the carrier strip is compressed and deformed. A portion of the at least one active agent is transferred from the carrier strip to the eyelashes, thereby retaining the curl or bend in the eyelashes.

In some implementations, the carrier strip has a fluid-activated composition including the at least one active agent thereon. In these implementations, the method further includes wetting the composition prior to positioning the eyelashes between the upper jaw and the lower jaw of the mechanical eyelash curler.

In some implementations, the carrier strip includes the at least one active agent therein. The carrier strip is configured to exude the least one active agent in response to being compressed and deformed.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1A is a perspective view of an eyelash curling device in a first position, according to some implementations of the present disclosure;

FIG. 1B is a perspective view of an unassembled eyelash curling device in a first position, according to other implementations of the present disclosure;

FIG. 2A is a perspective view of the eyelash curling device of FIG. 1A in a second position, according to some implementations of the present disclosure;

FIG. 2B is a perspective view of the eyelash curling device of FIG. 1B in an assembled, second position, according to some implementations of the present disclosure; and

FIG. 3 is a perspective view of a user curling her eyelashes using an eyelash curling device, according to some implementations of the present disclosure.

While the present disclosure is susceptible to various modifications and alternative forms, specific implementations have been shown by way of example in the drawings and will be described in further detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration. It should be understood that numerous specific details, relationships, and methods are set forth to provide a full understanding of the disclosure. One having ordinary skill in the relevant art, however, will readily recognize that the disclosure can be practiced without one or more of the specific details, or with other methods. In other instances, well-known structures or operations are not shown in detail to avoid obscuring the disclosure. The present disclosure is not limited by the illustrated ordering of acts or events, as some acts may occur in different orders and/or concurrently with other acts or events. Furthermore, not all illustrated acts or events are required to implement a methodology in accordance with the present disclosure.

The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. For example, substantially cylindrical means that the object resembles a cylinder, but can have one or more deviations from a true cylinder. The term “comprising” means “including, but not necessarily limited to”; it specifically indicates open-ended inclusion or membership in a so-described combination, group, series and the like.

As stated herein, keeping eyelashes that have been curled using, e.g., a mechanical eyelash curling device, curled for a prolonged period of time is challenging to achieve. Often, one curls his/her eyelashes in traditional ways but a short time later finds that his/her eyelashes did not remain curled. Conventional eyelash curlers simply curl the eyelashes by pressing a bar against a rubber strip to create a temporary bend or curl. Some eyelash curlers apply additional heat while curling, but this heating process poses a potential danger to consumers (especially if it's heated improperly) and is difficult to use.

To solve these and other problems, in some implementations, the present disclosure combines the process of curling eyelashes and setting in the curl with the use of a carrier strip. The carrier strip includes at least one active agent formulated to increase the length of time for curl retention. The at least one active agent provides a basis for a longer lasting curl. The carrier strip can be placed in an eyelash curler mechanism to be used while curling the eyelashes. At least a portion of the at least one active agent is transferred from the carrier strip to the eyelashes while the eyelashes are being curled.

The at least one active agent can be released and/or transferred from the carrier strip to the eyelashes using various mechanism, including compression and/or the moistening of the carrier strip. Thus, the carrier strip can be used with or without a moisturizing step. In some embodiments, a final solution may be applied to the eyelashes after curling to further aid in curl retention.

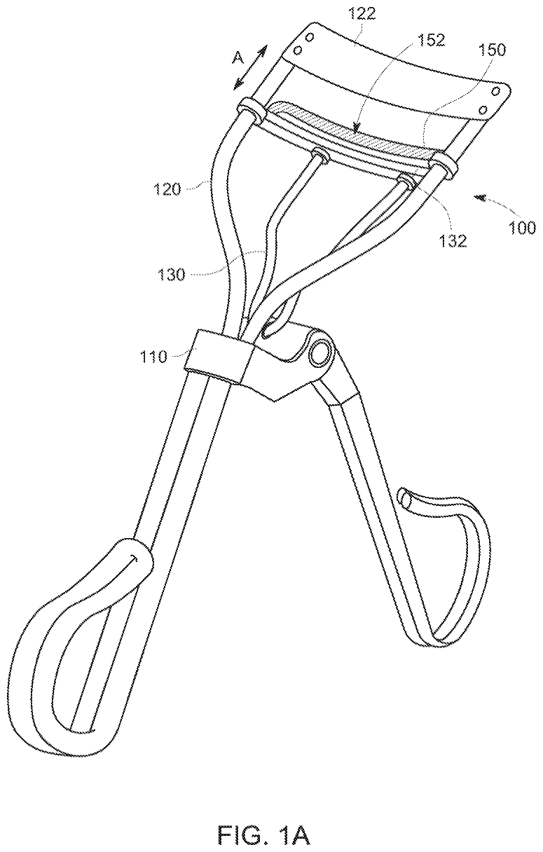

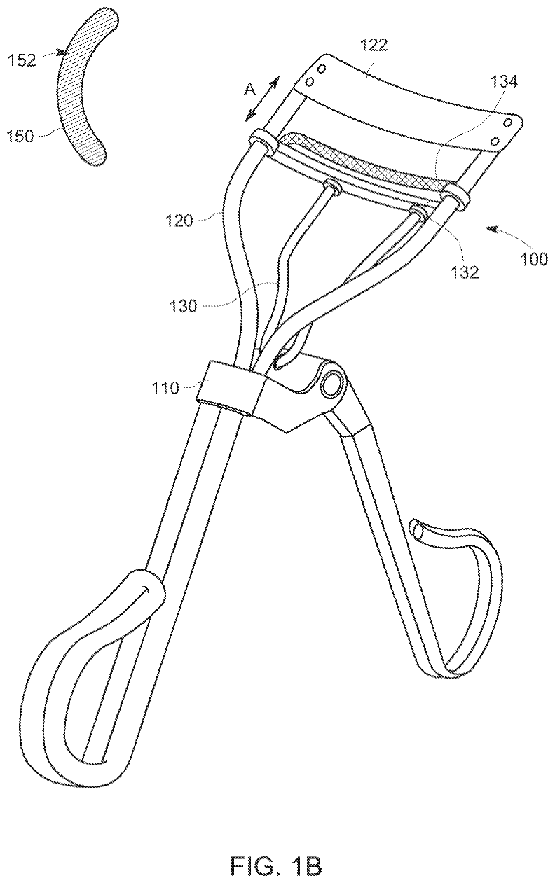

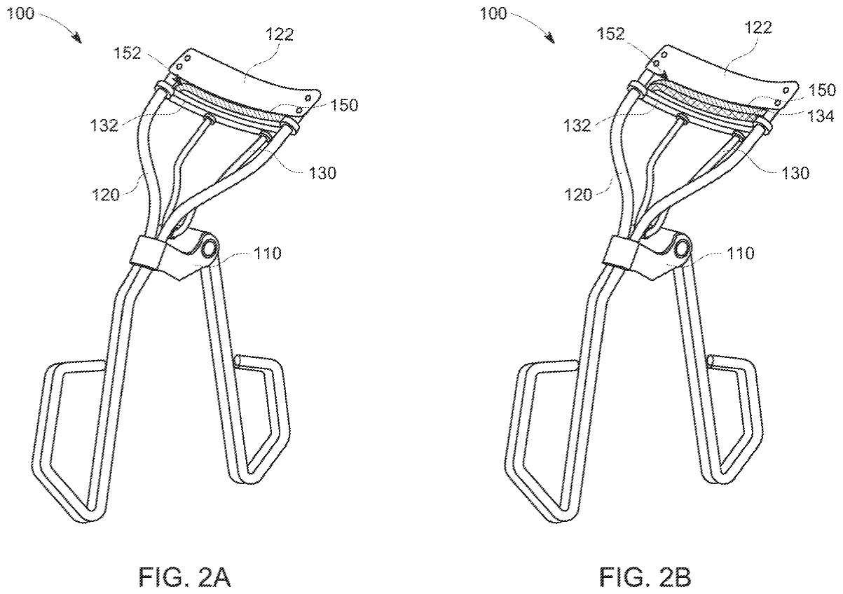

Referring generally to FIGS. 1A-2B, a non-limiting example of an eyelash curling device 100 is depicted. The eyelash curling device 100 includes a mechanical eyelash curler 110 and a carrier strip 150. The carrier strip 150 can include a blend of materials designed to provide the desired shape, physical characteristics (e.g., hardness and/or durometer value), one or more target agents, any combination thereof, or the like. In one non-limiting example, the carrier strip 150 may include a blend of carrier materials, filler materials, active agents, or any combination thereof.

The mechanical eyelash curler 110 includes a first portion 120 and a second portion 130. The first portion 120 and the second portion 130 are configured to move relative to one another. The first portion includes an upper jaw 122. The second portion includes a lower jaw 132. Movement of the first portion 120 and the second portion 130 relative to one another causes the upper jaw 122 and the lower jaw 132 to move from a first position (e.g., FIGS. 1A-1B) and a second position (e.g., FIGS. 2A-2B) and vice versa. In the first position, the upper jaw 122 and the lower jaw 132 are located at a distance from one another. Thus, when in the first position, the user may position his/her eyelashes between the upper and lower jaws 122, 132. In the second position, the upper jaw 122 and the lower jaw 132 are in contact with one another via the carrier strip 150. Thus, when in the second position, the user's eyelashes may be “sandwiched” between the upper and lower jaws 122, 132.

The carrier strip 150 can be coupled to the upper jaw 122 or the lower jaw 132 of the mechanical eyelash curler 110, either directly or indirectly. As shown in FIG. 1A, in some implementations, the carrier strip 150 is directly coupled to the lower jaw 132 of the mechanical eyelash curler 110. In some such implementations, the carrier strip 150 can be removably and/or adhesively coupled to the lower jaw 132 of the mechanical eyelash curler 110.

As shown in FIG. 1B, in some implementations, the carrier strip 150 can be indirectly coupled to the lower jaw 132 of the mechanical eyelash curler 110 via a base strip 134 (e.g., the carrier strip 150 is directly coupled to the base strip 134, and the base strip 134 is directly coupled to the lower jaw 132 of the mechanical eyelash curler 110). The base strip 134 may be a conventional (e.g., rubber) strip used in existing eyelash curlers such that the carrier strip 150 may be retroactively applied thereon to enhance the function thereof. In some implementations, the carrier strip 150 can be removably and/or adhesively coupled to the base strip 134 of the mechanical eyelash curler 110.

Even though the eyelash curling device 100 is shown to include a single carrier strip 150, an eyelash curling device of the present disclosure can include two or more carrier strips. For example, in some implementations, an eyelash curling device includes the mechanical eyelash curler 110 and two carrier strips. A first carrier strip is coupled to the upper jaw 122 of the mechanical eyelash curler 110, and a second carrier strip is coupled to the lower jaw 132 of the mechanical eyelash curler 110. This embodiment may be desirable to apply the active agent on the top and bottom portion of the eyelashes (as will be described in more detail below), thereby further enhancing curl retention.

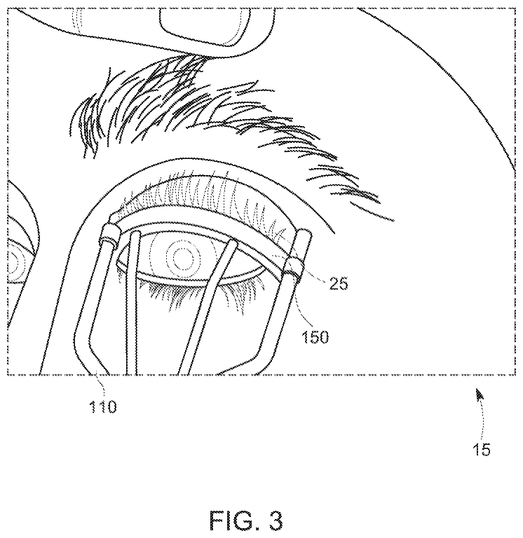

Thus, according to some implementations of the present disclosure, a method for retaining a curl or bend in eyelashes 125 of a user 15 begins with positioning the eyelashes of the user between the upper jaw 122 and the lower jaw 132 of the mechanical eyelash curler 110. Referring briefly to FIG. 3, a perspective view of a user 15 curling his/her eyelashes 25 using the eyelash curling device 100 is depicted. The upper jaw 122 and the lower jaw 132 of the mechanical eyelash curler 110 have been moved to the second position (e.g., the upper jaw 122 and the lower jaw 132 are in contact with the eyelashes 25 via the carrier strip 150). The eyelashes of the user are curled or bent by maintaining the mechanical eyelash curler 110 in the second position such that the carrier strip 150 is compressed and deformed. A portion of the at least one active agent 152 is transferred from the carrier strip 150 to the eyelashes, thereby retaining the curl or bend in the eyelashes.

According to the implementations described herein, and as will be described in more detail below, the carrier strip 150 includes at least one active agent 152 configured to aid in retaining a curl and/or bend in eyelashes 25 of a user 15 (depicted in FIG. 3). The at least one active agent 152 is formulated to increase a hold time of the curl and/or bend in any eyelashes. In some implementations, the at least one active agent 152 may also help to enhance a fullness of the eyelashes 25 by deposition of foundation materials to add body to the eyelashes 25. In some implementations, the carrier strip 150 may also include mascara for adding body and/or color to the eyelashes 25.

Thus, upon contact with the eyelashes 25, a portion of the at least one active agent 152 is transferred from the carrier strip 150 to the eyelashes 25. The transfer of the active agent 152 from carrier strip 150 to the eyelashes 25 aids in curling the eyelashes 25 and/or in retaining an existing curl in the eyelashes 25. In some implementations, the carrier strip 150 may also aid in mechanically curling the eyelashes by transferring pressure applied through the upper jaw 122 and/or the lower jaw 132.

It is contemplated that the carrier strip 150 may incorporate the at least one active agent 152 in any suitable ways. The carrier strip 150 can vary from including solely the at least one active agent 152, to being any of a variety of multi-component blends including the at least one active agent 152. For example, the carrier strip 150 can include the at least one active agent 152 therein or thereon.

In some implementations where the carrier strip 150 includes the he at least one active agent 152 thereon, the carrier strip 150 may be coated with a composition including the at least one active agent 152. The carrier strip 150 can be entirely coated with the composition containing the at least one active agent. Alternatively, only a portion of the carrier strip 150 may be coated with the composition including the at least one active agent (e.g., only a top layer or a bottom layer of the carrier strip 150 that is configured to contact the eyelashes is coated).

In some other implementations where the carrier strip 150 includes the at least one active agent 152 therein, a composition containing the at least one active agent 152 is incorporated into the material/matrix structure forming the carrier strip 150 and may be exuded therefrom using various suitable mechanisms, such as via pressure and/or contact with a second composition (e.g., a primer solution as disclosed herein). In some embodiments, the environment (e.g., temperature, humidity, etc.) may cause the active agent to be exuded from the carrier strip 150. The exudation process may include the composition “blooming” (or bleeding) to a surface of the carrier strip 150, such that the composition including the at least one active agent 152 can then be transferred to the eyelashes.

The “blooming” in the carrier strip 150 can be accelerated through mechanical action such as compression of the carrier strip 150 (e.g., pressure applied thereto by the upper jaw 122 and/or lower jaw 132 of the mechanical eyelash curler 110). For example, the carrier strip 150 can include a chlorinated wax in a silicone, which may promote the blooming effect. The carrier strip 150 in these implementations can further include one or more release regulating additives (e.g., crosslinkers, fillers, combinations thereof, or the like) configured to control the exudation of the at least one active agent.

In other implementations, the carrier strip 150 may be made of an absorbent and/or porous material with limited solubility (e.g., a sponge-like material). A composition including the at least one active agent 152 is non-soluble with respect to the carrier strip 150, but can be absorbed into the carrier strip 150. In response to pressure applied thereto, for example, by the upper jaw 122 and the lower jaw 132 in the second position (e.g., FIG. 2A), a portion of the composition including the at least one active agent 152 is released.

In some implementations, the carrier strip 150 may need to be moistened before the at least one active agent 152 can be transferred and/or released and applied to the eyelashes. In some such implementations, the composition including the at least one active agent 152 is dehydrated or dried, and/or is soluble (e.g., in water, alcohol, oil, emulsion, a combination thereof, or any other suitable solvent), whereas the carrier strip 150 is non-soluble.

The dehydrated composition of the carrier strip 150 can be re-hydrated in any suitable manner. For example, the dehydrated composition may be re-hydrated by applying a fluid (e.g., a primer solution) thereto. For example, a user may use a brush, a roller, a damp swab, a spray, or the like to apply the fluid to the dehydrated composition. In these implementations, either the eyelashes or the carrier strip 150 are pre-wetted, and some of the re-hydrated composition containing the at least one active agent 152 is then transferred to the eyelashes via contacting the carrier strip 150 to the eyelashes. The re-hydrated composition containing the at least one active agent 152 may then dry and form a film that will impart the desired “hold” for extended curl retention.

For example, the eyelashes 25 of the user 15 may be pretreated (e.g., pre-wetted) prior to being placed between the upper jaw 122 and the lower jaw 132 of the mechanical eyelash curler 110. The eyelashes 25 can be pretreated by coating the eyelashes 25 with a primer solution that wets the eyelashes. The primer solution may further promote wicking and/or transfer of the at least one active agent 152 from the carrier strip 150 to the eyelashes 25 when the carrier strip 150 is compressed and/or the carrier strip 150 contacts the eyelashes.

Although the eyelash curling device 100 is depicted in FIGS. 1A-3 as having the mechanical eyelash curler 110 and the carrier strip 150, an eyelash curling device of the present disclosure can have more or fewer components.

In one embodiment, an alternative eyelash curling device further includes a storage component for enclosing the carrier strip 150, such that the storage component inhibits the composition including the one or more active agents on the carrier strip 150 from dehydrating (e.g., losing a portion or all of the active agent 152). For example, in some implementations, the storage component can include a substantially airtight compartment (e.g., a cap, or any suitable airtight storage compartment). Additionally or alternatively, the carrier strip 150 is coupled to a removable seal or covering to protect the carrier strip 150 while not in use. This embodiment may be desirable because rehydration of the composition including the active agent (as discussed above) may not be needed. It is further contemplated that the eyelash curling device may include separate storage compartments for maintaining the carrier strip 150 having the hydrated composition in a substantially air-tight environment to reduce or eliminate dehydration.

The carrier strip 150 may comprise any suitable material. Preferably, the carrier strip 150 is formed of a material that is at least somewhat bendable or flexible. In some embodiments, the carrier strip 150 is inflexible and/or rigid. In some embodiments, the carrier strip 150 is flexible. In some embodiments, the carrier strip 150 includes a polymer or combination of polymers. In other embodiments, the carrier strip 150 can be made of any known elastomeric material, thermoplastic elastomeric material, or any combination thereof, including, but not limited to, silicones, natural rubbers, synthetic organic rubbers, or the like. Other examples of such materials can be any of a number of thermoplastics, such as, but not limited to, nylons, polyolefins, a combination thereof, or the like. Further examples of such materials include styrene-isoprene-styrene copolymers, synthetic polyisoprenes, a combination thereof, or the like.

The composition containing the at least one active agent 152 may comprise a film formers, fixatives safe for human contact, cosmetic adhesives, medical adhesives, or any combination thereof, including, but not limited to, vinyl pyrrolidones, polyvinylpyrrolidones (“PVP”), PVP/vinyl acetates, PVP/dimethylaminoethyl methacrylate copolymers, poly acrylates (such as EUDRAGIT® series by Evonik Industries AG, Essen, North Rhine-Westphalia, Germany), esters of acrylic and methacrylic acids, acrylates/hydroxyesters acrylates copolymers (ACUDYNE™ DHR by Dow Corning, Midland, Mich.), polyvinylalcohol, polyvinylacetate, polyacrylates, polyethylene glycol s/polyol s, silicones/waxes, biopolymers (e.g., carrageenans), cellulosics (e.g., hypromellose, carboxymethyl cellulose, hydroxyethyl cellulose), polyquaternium-69, octylacrylamide/acrylates/butylaminoethyl methacrylate copolymer, vinyl caprolactam/vinylpyrrolidone/dimethylaminoethyl methacrylate copolymer, isobutylene/dimethylaminopropyl maleimide/ethoxylated maleimide/maleic acid copolymer (e.g., Aquaflex™ XL 30 by Ashland, Covington, Ky.), guar hydroxypropyltrimonium chloride, methacrylic acid/sodium acrylamidomethyl propane sulfonate copolymer, chitosans, polyacrylamide polymers, polyacrylic acids, gums, mastics, tackifiers, sulfopolyesters, sodium polyitaconate, octylacrylamide/acrylates/butylaminoethyl methacrylate copolymer, carbomers, polyurethanes, adenosine monophosphate-acrylates copolymers, acrylates crosspolymers, carbopols, natural and synthetic waxes.

In some non-limiting implementations, the composition including the at least one active agent 152 includes film formers, such as polyethylene glycols, isobutylene/dimethylaminopropyl maleimide/ethoxylated, maleimide/maleic acid copolymers, or any combination thereof. The polyethylene glycols may have varying molecular weight. The quantities and/or ratios can be optimized, e.g., according to environmental parameters.

In those implementations where the carrier strip 150 needs to be moistened/re-hydrated, an exemplary primer solution for the pre-wetting is water. In some such implementations, the ambient humidity of the atmosphere and/or the eyelashes may suffice to activate and/or transfer a portion of the at least one active agent 152. For example, the carrier strip 150 may include a “dry” adhesive, such as a tackifier and/or a pressure sensitive agent. The fluid-activated composition can be formulated according to a climate of a target customer base. Another exemplary primer solution is a lipophilic material (e.g., oil). The lipophilic material aids in the extraction of the active agent 152 from the carrier strip 150.

Therefore, there may be several options for use depending on the components of the eyelash curling device. A first option includes wetting the eyelashes before curling the eyelashes with the combination of the mechanical eyelash curler and the carrier strip. A second option includes wetting the carrier strip before using the combination of the mechanical eyelash curler and the carrier strip. According to a third option, a protective barrier or covering is first removed (e.g., peeled away) from the carrier strip, and then the carrier strip is used in combination with the mechanical eyelash curler to perform a curl. According to a fourth option, the carrier strip may be manufactured to perform with the action of curling without the need for any further wetting steps (e.g., in embodiments where the composition including the one or more active agents is incorporated within the carrier strip). An additional storage compartment or component may or may not be used to aid in the preservation of the carrier strip before use with the mechanical eyelash curler.

The carrier strip in accordance with the embodiments described herein may be manufactured and/or sold by itself, e.g., as a plurality of reusable or disposable carrier strips, or with a mechanical eyelash curler (either pre-assembled or to be assembled by a user). The carrier strip may be manufactured as a single-use item, or a multi-use item (e.g., can be used until all of the at least one active agent is released/transferred away from the carrier strip). In some implementations where the carrier strip can be used multiple times, the carrier strip may include an indicator for indicating to a user when the active agent has been used up or is close to being use dup. For example, a color of the carrier strip can change to indicate a remaining amount of the at least one active agent in the carrier strip.

One of the advantages of the present disclosure is that the at least one active agent can be applied to a user's eyelashes at the time of curling, as opposed to first applying the at least one active agent then curling, which may be analogous to putting on hairspray before styling your hair. The present disclosure allows for the fixative to form in the shape of the curl, as opposed to potentially working against it when applied before. For example, application of an active agent prior to curling may cause the lashes to become tacky or sticky, thereby causing the lashes to stick to the eyelash curler during the curling process, which may be painful or even cause the undesired removal of lashes.

Another advantage of the present disclosure is that potential harm to the user is reduced because the at least one active agent is not directly applied to eyelashes of a user. Instead of directly applying to the eyelashes, thus potentially inadvertently causing the at least one active agent to enter the eyes of the user or poking a user's eye during application, a portion of the at least one active agent is transferred from the carrier strip to the eyelashes of the user upon pressure and/or contact. Put another way, the embodiments described herein reduce the risk of harm to a user by applying the active agent to the lashes at the same time as curling the lashes (e.g., in a single step), thereby eliminating at least one process that involves putting the user's eye in harm's way. This may also be beneficial because it reduces the time required to curl the eyelashes.

Yet a further advantage of the present disclosure is that some implementations disclosed herein do not require adding any steps to conventional eyelash curling to enhance/maintain an eyelash curl. Even in the implementations where pre-wetting is required, it only adds one additional step consisting of a few seconds of time—at no added danger/risk of eye injury to the user, while the eyelashes can remain curled for longer than using the conventional eyelash curler.

In some other implementations, the carrier strip does not contain the at least one active agent. Instead, the primer solution (e.g., to be coated on the eyelashes of the user and/or on the carrier strip) includes the at least one active agent. For example, a user may use a brush, a roller, a damp swab, a spray, or the like to apply the primer solution including the at least one active agent to the eyelashes and/or the carrier strip. The primer solution can include a dilute aqueous mixture of a film forming polymer, which would wet and pre-deposit the at least one active agent.

While various examples of the present disclosure have been described above, it should be understood that they have been presented by way of example only, and not limitation. Numerous changes to the disclosed examples can be made in accordance with the disclosure herein without departing from the spirit or scope of the disclosure. Thus, the breadth and scope of the present disclosure should not be limited by any of the above described examples. Rather, the scope of the disclosure should be defined in accordance with the following claims and their equivalents.

Although the disclosure has been illustrated and described with respect to one or more implementations, equivalent alterations and modifications will occur to others skilled in the art upon the reading and understanding of this specification and the annexed drawings. In addition, while a particular feature of the disclosure may have been disclosed with respect to only one of several implementations, such feature may be combined with one or more other features of the other implementations as may be desired and advantageous for any given or particular application.

The terminology used herein is for the purpose of describing particular examples only and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this disclosure belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

What is claimed is:

1. An eyelash curling device, comprising:
a mechanical eyelash curler including a first portion and a second portion configured to move relative to one another, the first portion including an upper jaw, the second portion including a lower jaw, wherein the movement of the first and second portions relative to one another causes the upper and lower jaws to move from a first position in which the upper and lower jaws are located at a distance from one another and a second position in which the upper and lower jaws are in contact with one another; and
a carrier strip coupled to at least one of the upper and lower jaws, the carrier strip including at least one active agent therein or thereon, the at least one active agent being configured to aid in retaining a curl or bend in eyelashes of a user when the at least one active agent contacts the eyelashes.

2. The eyelash curling device of claim 1, wherein the carrier strip comprises a flexible material having the at least one active agent in a matrix of the flexible material.

3. The eyelash curling device of claim 2, wherein the carrier strip is configured to exude the least one active agent in response to pressure applied thereto by the upper and lower jaws in the second position.

4. The eyelash curling device of claim 1, wherein the at least one active agent is a non-soluble fixative agent.

5. The eyelash curling device of claim 1, wherein the carrier strip is formed of an absorbent material configured to release a portion of the at least one active agent in response to pressure applied thereto by the upper and lower jaws in the second position.

6. The eyelash curling device of claim 1, wherein the carrier strip is a flexible material having a fluid-activated composition including the at least one active agent thereon.

7. The eyelash curling device of claim 6, wherein the fluid is water.

8. The eyelash curling device of claim 1, further comprising a substantially airtight compartment for enclosing the carrier strip, wherein the carrier strip includes a solution comprising the at least one active agent thereon.

9. The eyelash curling device of claim 1, further comprising a base strip coupled to at least one of the upper and lower jaws, wherein the carrier strip is coupled to the base strip.

10. The eyelash curling device of claim 9, wherein the carrier strip is removably coupled to the base strip.

11. The eyelash curling device of claim 9, wherein the carrier strip is adhesively coupled to the base strip.

12. The eyelash curling device of claim 1, wherein the carrier strip includes a flexible material at least partially coated with the at least one active agent.

13. The eyelash curling device of claim 1, wherein the carrier strip includes is a styrene-isoprene-styrene copolymer or a synthetic polyisoprene.

14. A carrier strip for use in enhancing or maintaining an eyelash curl, the carrier strip comprising:
a flexible base material; and
at least one active agent positioned on or within the base material, the act least one active agent configured to aid in retaining the eyelash curl when the at least one active agent contacts the eyelashes,
wherein the carrier strip is configured to couple to at least one of an upper jaw and a lower jaw of a mechanical eyelash curler, the mechanical eyelash curler including a first portion and a second portion configured to move relative to one another, the first portion including the upper jaw, the second portion including the lower jaw, wherein the movement of the first and second portions relative to one another causes the upper and lower jaws to move from a first position in which the upper and lower jaws are located at a distance from one another and a second position in which the upper and lower jaws are in contact with one another via the carrier strip.

15. The carrier strip of claim 14, wherein the flexible base material includes a matrix having the at least one active agent therein, wherein the carrier strip is configured to exude the least one active agent in response to pressure applied thereto by the upper and lower jaws in the second position.

16. The carrier strip of claim 14, further comprising a fluid-activated composition including the at least one active agent therein, the fluid-activated composition being applied on a surface of the flexible base material.

17. The carrier strip of claim 16, wherein the fluid is water.

18. A method for retaining a curl or bend in eyelashes of a user, the method comprising:
positioning the eyelashes of the user between an upper jaw and a lower jaw of a mechanical eyelash curler, the mechanical eyelash curler including an upper jaw and a lower jaw, at least one of the upper and lower jaws having a carrier strip coupled thereto, the carrier strip including at least one active agent therein or thereon, the at least one active agent being configured to aid in retaining a curl or bend in eyelashes of a user when the at least one active agent contacts the eyelashes;
moving the upper jaw and the lower jaw of the mechanical eyelash curler from a first position in which the upper and lower jaws are located at a distance from one another to a second position in which the upper and lower jaws are in contact with one another and the eyelashes and are sandwiched therebetween, the eyelashes contacting the carrier strip;
curling or bending the eyelashes of the user by maintaining the mechanical eyelash curler in the second position such that the carrier strip is compressed and deformed; and
transferring a portion of the at least one active agent from the carrier strip to the eyelashes, thereby retaining the curl or bend in the eyelashes.

19. The method of claim 18, wherein the carrier strip has a fluid-activated composition including the at least one active agent thereon, and wherein the method further comprises wetting the composition prior to positioning the eyelashes between the upper jaw and the lower jaw of the mechanical eyelash curler.

20. The method of claim 18, wherein the carrier strip includes the at least one active agent therein, the carrier strip being configured to exude the least one active agent in response to being compressed and deformed.
